SEQUENCE LISTING (6 sequences)

SEQ ID NO: 1 - DNA, 21 bp, Artificial Sequence
  Beta-actin forward primer
ggattcctatgtgggcgacga

SEQ ID NO: 2 - DNA, 21 bp, Artificial Sequence
  Beta-actin reverse primer
cgctcggtgaggatcttcatg

SEQ ID NO: 3 - DNA, 20 bp, Artificial Sequence
  COL1A1 forward primer
agggccaagacgaagacatc

SEQ ID NO: 4 - DNA, 21 bp, Artificial Sequence
  COL1A1 reverse primer
agatcacgtcatcgcacaaca

SEQ ID NO: 5 - DNA, 24 bp, Artificial Sequence
  MMP1 forward primer
tctgacgttgatcccagagagcag

SEQ ID NO: 6 - DNA, 23 bp, Artificial Sequence
  MMP1 reverse primer
cagggtgacaccagtgactgcac

(19) United States
(12) Patent Application Publication
(10) Pub. No.: US 2016/0067522 A1
(43) Pub. Date: Mar. 10, 2016

(54) METHOD FOR SKIN IMPROVEMENT USING LOW-DOSE RADIATION

(71) Applicant: KONKUK UNIVERSITY INDUSTRIAL COOPERATION CORPORATION, Seoul (KR)

(72) Inventors: SUNG-KWAN AN, Seoul (KR); Seung-Hee Bae, Seoul (KR); Hwa-Jun Cha, Gyeonggi-do (KR); Ka-Ram Kim, Seoul (KR); Sang-Hun Shin, Chungcheongbuk-do (KR); Hyun-Hee Jang, Gyeonggi-do (KR)

(21) Appl. No.: 14/839,766

(22) Filed: Aug. 28, 2015

(30) Foreign Application Priority Data
Sep. 4, 2014 (KR) 10-2014-0117780

(51) Int. Cl. A61N 5/06; A61N 5/10

(52) U.S. Cl. CPC A61N 5/0616; A61N 5/10; A61N 2005/1087; A61N 2005/1089; A61N 2005/0661

(57) ABSTRACT

The present disclosure relates to a method for skin improvement using low-dose radiation, and the low-dose radiation can exhibit excellent effects of improving skin wrinkles, improving skin barriers, improving skin thickness, improving skin elasticity, and improving skin moisture through the mechanisms of promoting the growth of normal human dermal fibroblasts, increasing the collagen synthesis within the normal human dermal fibroblast, or reducing the metalloproteinase synthesis within the normal human dermal fibroblasts, thus being effectively used for skin improvement. Additionally, the low-dose radiation of the present disclosure can exhibit excellent hair growth-promoting effect through the mechanisms of promoting the growth of human dermal papilla cells or activating hair follicle cells, thus being useful for the improvement of promoting hair growth.

### CROSS-REFERENCE TO RELATED APPLICATIONS

This application is based on and claims priority from Korean Patent Application No. KR2014-0117780, filed on Sep. 4, 2014, with the Korean Intellectual Property Office, the disclosure of which is incorporated herein in its entirety by reference.

### TECHNICAL FIELD

The present disclosure relates to a method for skin improvement using low-dose radiation.

### BACKGROUND

Skin is located on the outermost part of the body and plays the most fundamental and important role of protecting the body against various external factors. For this purpose, skin consists of various layers and cells. Skin can be largely divided into an epidermal layer and a dermal layer. The epidermal layer consists of keratinocytes, melanocytes, and immunocytes, and plays a role of skin barriers, whereas the dermal layer includes dermal fibroblasts which play the roles of controlling elasticity and wrinkles of skin.

In the epidermal layer, keratinocytes present in the basal membrane continue to repeat the processes of proliferation and differentiation in a balanced manner and thereby establish a layered structure consisting of a basal layer, a spinous layer, a granular layer, and a horny layer. The keratinocytes, while being separated from the basal layer passed through the spinous layer and the granular layer, dissipate organelles and nucleoplasm in the nucleus and undergo keratinization, where they become filled with keratin and finally converted into corneocytes, and bind with other corneocytes through intercellular lipids thereby forming a horny layer of skin. The thus-formed horny layer plays a role to protect the skin from various external stimuli and prevent the evaporation of water from the body. However, as the horny layer becomes excessively detached due to environmental reasons and repeated facial washes, or the proliferation and differentiation of keratinocytes may not be proceeded normally due to skin aging, thus being unable to serve the function as skin barriers thereby becoming vulnerable to various skin diseases.

The dermal fibroblasts present in the dermal layer express collagen and elastin to thereby render elasticity and flexibility to skin, and are thus the most influential factor regarding skin wrinkles. There are 14 different types in collagen, and among them, type I collagen is most abundant in skin. Collagen is formed by two α1 chains and one α2 chain twisted in a triple helical shape, and these chains are expressed from Col1A1 and Col1A2 genes. Collagen produced in cells is excreted extracellularly, combined with each other in the extracellular matrix, and form a collagen fiber. The collagen synthesis becomes deteriorated along with the aging process or by the external environment such as UV, and the increase in the activity of matrix-metalloproteases, a collagen decomposing enzyme, promotes the collagen decomposition thereby lowering elasticity and flexibility. As such, the skin problems occurring in the epidermal layer and the dermal layer cannot be solved by cosmetics such as humectants, and it is necessary to restore the skin so that keratinocytes and dermal fibroblasts, which serve very important roles in each respective skin region, can perform their own functions.

Additionally, follicles and hairs are maintained while going through with the life cycle, which consist of a growth phase, a regressing phase, and a quiescent phase, and repeating growth and hair-loss. However, people with alopecia have hairs mostly at in their quiescent phase, where papillas stop their activities on the scalp and allow hairs to stay on the scalp, and thus apparently displaying the phenomenon of hair-loss. Examples of hair growth agents to solve the hair-loss problem are only Minoxidil® and Finasteride® approved by US FDA. However, Minoxidil® reportedly has a sticky feeling and skin irritations, whereas Finasteride® has been reported to have an adverse effect of sexual dysfunction. Additionally, these agents are known effective only when they are continuously administered and thus there is inconvenience in administration compliance.

Since the discovery of radiation, high-dose radiation has been used in medical field as one of the three major treatments along with surgeries and chemical therapies. However, let alone the high-dose radiation, the living organisms on earth are already exposed to radioactive isotopes from natural radiation generated from the earth's crust, air, or space crafts, and the amount of exposure is about 1 msv per year, although it varies from region to region. In this regard, the importance of the study on the effect of low-dose radiation on human bodies has been acknowledged but the exact mechanism of how the low-dose radiation affects human bodies or how it may be used in the industries still remain to be elucidated.

Low dose radiation (LDR) refers to low-intensity radiation close to that in nature. Although a large amount of radiation may harm living organisms, a small amount of low-dose radiation has been reported to promote physiological activities of living organisms thus giving advantageous effects, such as prolonging life expectancy or promoting growth or lowering the occurrence rate of tumors. This is called radiation hormesis. The low-dose radiation in the range of milligray (mGy), unlike the high-dose radiation, reportedly can induce resistance to diseases and exhibit an effect of adaptive protection (Sakai K., et al., Dose Response, 2006, 4(4):327 to 332; Sakai K., et al, Int. J. Low Radiat., 2003, 1(1):142 to 146; Scott BR., Dose Response, 2008 6(3):299 to 318), and there was also a report that the irradiation of a certain amount of low-dose radiation on experimental mice with diabetes genetic factor exhibited an improvement in symptoms (Sakai K., et al., Dose Response, 2006, 4(4):327 to 332). Radiation in the amount of 0.25 Gy or less is known to prevent DNA damage and repair damaged DNA (Le XC., et al., Science, 1998, 280(5366):1066 to 1069), and low-dose radiation is known to have an immune-strengthening effect (Nogami M., et al., Int. J. Radiat. Biol., 1993, 63(6):775 to 783; Nogami M., et al., Radiat. Res., 1994, 139(1):47 to 52). However, there has been no report regarding the effect of irradiation of low-dose radiation on skin improvement.

### PRIOR ART DOCUMENT

### Patent Document

### Korean Patent Application Publication No. 10-2011-0051850

### SUMMARY

Accordingly, the present inventors, while studying effective technologies regarding skin improvement, discovered by detailed confirmation through the epidermal layer and dermal layer of skin that the irradiation of low-dose radiation can be effectively used for skin improvement thereby completing the present disclosure.

Accordingly, an object of the present disclosure is to provide a method of skin improvement using low-dose radiation.

Additionally, another object of the present disclosure is to provide a method of promoting hair growth using low-dose radiation.

The foregoing summary is illustrative only and is not intended to be in any way limiting. In addition to the illustrative aspects, embodiments, and features described above, further aspects, embodiments, and features will become apparent by reference to the drawings and the following detailed description.

In order to achieve the above objects, the present disclosure provides a method for skin improvement including the irradiation of low-dose radiation.

In an exemplary embodiment of the present disclosure, the low-dose radiation may be at least one selected from the group consisting of α-ray, β-ray, γ-ray, electron-ray, UV-ray, and X-ray.

In an exemplary embodiment of the present disclosure, the low-dose radiation may be irradiated ranging from 0.01 Gy to 0.2 Gy.

In an exemplary embodiment of the present disclosure, the low-dose radiation may be irradiated ranging from 0.01 Gy to 0.1 Gy.

In an exemplary embodiment of the present disclosure, the low-dose radiation may be irradiated ranging from 0.05 Gy to 0.1 Gy.

In an exemplary embodiment of the present disclosure, in irradiating the low-dose radiation, the low-dose radiation may be irradiated once or a multiple times at predetermined intervals.

In an exemplary embodiment of the present disclosure, in irradiating the low-dose radiation, the growth of normal human dermal fibroblast may be promoted, the collagen synthesis within the normal human dermal fibroblast may be increased, or the metalloproteinase synthesis within the normal human dermal fibroblast may be decreased.

In an exemplary embodiment of the present disclosure, in irradiating the low-dose radiation, the growth of keratinocytes is promoted or the differentiation of keratinocytes is promoted.

In an exemplary embodiment of the present disclosure, the skin improvement may be selected from the group consisting of improvement of skin wrinkles, improvement of skin barriers, improvement of skin thickness, improvement of skin elasticity, and improvement of skin moisture.

Additionally, the present disclosure further provides a method of promoting hair growth including the irradiation of low-dose radiation.

In an exemplary embodiment of the present disclosure, the low-dose radiation may be at least one selected from the group consisting of α-ray, β-ray, γ-ray, electron-ray, UV-ray, and X-ray.

In an exemplary embodiment of the present disclosure, the low-dose radiation may be irradiated ranging from 0.01 Gy to 0.2 Gy.

In an exemplary embodiment of the present disclosure, the low-dose radiation may be irradiated ranging from 0.01 Gy to 0.1 Gy.

In an exemplary embodiment of the present disclosure, the low-dose radiation may be irradiated ranging from 0.05 Gy to 0.1 Gy.

In an exemplary embodiment of the present disclosure, in irradiating the low-dose radiation, the growth of human dermal papilla cells may be promoted or hair follicle cells may be activated.

### ADVANTAGEOUS EFFECT OF THE INVENTION

The low-dose radiation of the present disclosure can exhibit excellent effects of improving skin wrinkles, improving skin barriers, improving skin thickness, improving skin elasticity, and improving skin moisture through the mechanisms of promoting the growth of normal human dermal fibroblast, increasing the collagen synthesis within the normal human dermal fibroblast, or reducing the metalloproteinase synthesis within the normal human dermal fibroblast, thus being effectively used for skin improvement.

Additionally, the low-dose radiation of the present disclosure can exhibit excellent hair growth-promoting effect through the mechanisms of promoting the growth of human dermal papilla cells or activating hair follicle cells, thus being useful for the improvement of promoting hair growth.

### BRIEF DESCRIPTION OF THE DRAWINGS

FIG. 1 illustrates the results confirming the effect of promoting growth of normal human dermal fibroblast (nHDF) by the irradiation of low-dose radiation.

FIG. 2 illustrates the results confirming the effect of increasing the expression of Col1A1 by the irradiation of low-dose radiation.

FIG. 3 illustrates the results confirming the effect of decreasing the expression of MMP1 by the irradiation of low-dose radiation.

FIG. 4 illustrates the results confirming the effect of improving skin wrinkles in mice by the irradiation of low-dose radiation.

FIG. 5 illustrates the results confirming the effect of improving skin thickness in mice by the irradiation of low-dose radiation.

FIG. 6 illustrates the results confirming the effect of improving skin elasticity in mice by the irradiation of low-dose radiation.

FIG. 7 illustrates the results confirming the effect of growing keratinocytes by the irradiation of low-dose radiation.

FIG. 8 illustrates the photograph confirming the effect of promoting differentiation of keratinocytes by the irradiation of low-dose radiation.

FIG. 9 illustrates the results confirming the effect of recovering skin barriers by the irradiation of low-dose radiation.

FIG. 10 illustrates the results confirming the effect of improving skin texture in mice by the irradiation of low-dose radiation.

FIG. 11 illustrates the results confirming the effect of growing human dermal papilla cells by the irradiation of low-dose radiation.

FIG. 12 illustrates the results confirming the effect of promoting hair growth in mice by the irradiation of low-dose radiation.

### DETAILED DESCRIPTION

In the following detailed description, reference is made to the accompanying drawings, which form a part hereof. The illustrative embodiments described in the detailed description, drawing, and claims are not meant to be limiting. Other embodiments may be utilized, and other changes may be made, without departing from the spirit or scope of the subject matter presented here.

The present disclosure is characterized in that low-dose radiation is used for skin improvement and promotion of hair growth.

The low-dose radiation is preferably at least one selected from the group consisting of α-ray, β-ray, γ-ray, electron-ray, UV-ray, and X-ray, and more preferably γ-ray, but is not limited thereto.

The preferable range of the low-dose radiation is from 0.01 Gy to 0.2 Gy, more preferably from 0.01 Gy to 0.1 Gy, and most preferably from 0.05 Gy to 0.1 Gy, but is not limited thereto.

In the present disclosure, “low-dose radiation” is defined as radiation in the amount of 0.1 Gy (100 mSv) or less, and this definition refers to the criteria for low-dose radiation, 0.1 Gy (100 mSv), defined in BIOLIGICAL MECHANISMS OF RADIATION ACTIONS AT LOW DOSES (page 2) published in 2012 at United Nations Scientific Committee on the Effect of Atomic Radiation (UNSCEAR).

In the present disclosure, gray (Gy) is an SI unit regarding absorbed radiation dose, and 1 Gy refers to energy absorption of one joule per 1 kg of a material. This is the same unit as sievert (Sv). In order to avoid the risk due to any confusion between the absorbed dose and the irradiated dose, Sv is used for the irradiation dose while Gy is used for the absorbed dose. In the present disclosure, Gy was used as a basic unit with reference to the radiation being absorbed.

1 Gy=1 J/kg=1 m²·s⁻²

As used herein, the term “radiation exposure” refers to irradiating radiation to a material, and a sample is irradiated by radioactive isotopes or radiation from an accelerator, or irradiating neutrons or γ-ray within a nuclear reactor. The γ-ray is currently used in radiation chemistry, food sterilization, medical therapies or a non-destructive test such as radiography. The radiation exposure of neutrons is widely used in behavioral studies of fuels and materials in the reactor or activation analysis, and preparation of radioactive isotopes, and in the present disclosure, radiation exposure was used for skin improvement.

In the present disclosure, for the irradiation of radiation, cesium-137 medium-dose radiation irradiator (MDI-KIRAMS 137) manufactured by Korea Institute of Radiological and Medical Sciences (KIRAMS) was used as an irradiator. The radiation type used was cesium-137 γ-ray, and the radiation dose rate was 0.67 cGy/min. The method of irradiation was Total Body Irradiation (TBI).

According to a preferred embodiment of the present disclosure, “skin improvement” is characterized to be selected from improvement of skin wrinkles, improvement of skin barriers, improvement of skin thickness, improvement of skin elasticity, and improvement of skin moisture, but is not limited thereto, and includes the effect of promoting hair growth. Skin improvement refers to increasing of physiological functions of facial skin and entire skin of the body and activating metabolism via physical methods, chemical methods, and the like, to thereby supply nutrients thereto, as a result, enabling to maintain beautiful skin, protect and improve skin and manage skin. The purposes of skin improvement are to recover the physiological functions of skin thereby maintaining the skin in a healthy and elastic state, helping the skin to recover its original properties and characteristics to be improved to a normal skin and maintain healthy skin.

The method of skin improvement of the present disclosure includes the improvement in skin wrinkles. The method of the present disclosure exhibits stable and excellent effects of improving skin wrinkles via low-dose radiation, and this is clearly described in Examples below. The improvement of skin wrinkles as a use of the present disclosure should be understood to include the conventional uses of skin protection (prevention of wrinkles, removal of wrinkles, and the like).

Additionally, the method of skin improvement including the irradiation of low-dose radiation of the present disclosure works very effectively on the improvement of skin barriers. The keratinocytes in the basal layer go through with differentiation and migration processes to form a new horny layer, and this entire process is called keratinization cycle, which takes about 28 days. The keratinocytes which became aged during the process will be detached, and the skin will be replaced with new horny layer. When the differentiation rate becomes slow by the deterioration in keratinocytes according to skin aging, the replacement with a new horny layer cannot be smoothly performed thus making the skin rough and deteriorating the function as a barrier. The method of the present disclosure exhibits stable and excellent effects of improving the function as a skin barrier via low-dose radiation, and was confirmed to have the effects of promoting the growth of keratinocytes, promoting the differentiation of keratinocytes, and recovering the mouse skin barriers, and these are clearly described in Examples below.

Additionally, the method of skin improvement including the irradiation of low-dose radiation of the present disclosure works very effectively on the improvement of skin thickness. When collagen and elastin present in the dermal layer become reduced, the thickness of the dermal layer becomes reduced, and the method of the present disclosure exhibits stable and excellent effect of improving skin thickness via low-dose radiation. The effect of improving skin thickness by low-dose radiation was confirmed by measuring the dermal layer thickness of a mouse using a skin ultrasound measuring device, and this is clearly described in Examples below.

Additionally, the method of skin improvement including the irradiation of low-dose radiation of the present disclosure works very effectively on the improvement of skin elasticity. The increase of collagen produced by dermal fibroblasts and the decrease of metalloproteinase can affect the skin elasticity, and the method of the present disclosure exhibits stable and excellent effect of improving skin elasticity via low-dose radiation. The effect of improving skin elasticity via low-dose radiation was confirmed by measuring the degree of change in skin elasticity of a mouse, and this is clearly described in Examples below.

Additionally, the method of skin improvement including the irradiation of low-dose radiation of the present disclosure works very effectively on the promotion of hair growth. As used herein, the term “promotion of hair growth” has the same meaning as the promotion of hair nurturing or hair cultivation, which are the terms also used in the art. The method of the present disclosure exhibits stable and excellent effect of promoting hair growth via low-dose radiation. The effects of promoting growth of human dermal papilla cells and activating follicle cells via low-dose radiation were confirmed, and these are clearly described in Examples below.

As used herein, the phrase “exposing once or multiple times at predetermined time intervals” refers to irradiating until the amount of radiation reaches the total amount of radiation, without being limited to the number of irradiations of radiation.

The advantages and characteristics of the present disclosure, and the methods to achieve the same will become apparent by referring to the examples described herein below. Hereinafter, the present disclosure will be described in more detail with reference to the following examples. However, the following examples are provided for illustrative purposes only, and the scope of the present disclosure should not be limited thereto in any manner.

### Example 1

### Effect of Promoting Growth of Normal Human Dermal Fibroblast by Low-Dose Radiation

In order to confirm the effect of skin improvement by low-dose radiation, the present inventors examined whether low-dose radiation can promote the growth of fibroblasts, which synthesize collagen, a constituting component of dermis. Normal human dermal fibroblasts (nHDF) were purchased from Lonza (Switzerland) for use. For a cell culture solution, Dulbeco's modified Eagle's medium (DMEM; Gibco, USA) was added with 10% fetal bovine serum (FBS; Gibco), 10% penicillin (100 unit/mL), and streptomycin (100 g/mL) for use. Culture conditions were 37° C., 95% humidity, and 5% CO₂ incubator, and cells from 3 to 10 generations were used in the experiments.

In order to examine the level of cytotoxicity generated when normal human dermal fibroblasts (nHDF) were exposed to low-dose radiation, normal human dermal fibroblasts were irradiated with 0 Gy, 0.01 Gy, 0.05 Gy, and 0.1 Gy, cultured for 24, 48, and 72 hours, and the viability of the cells were measured.

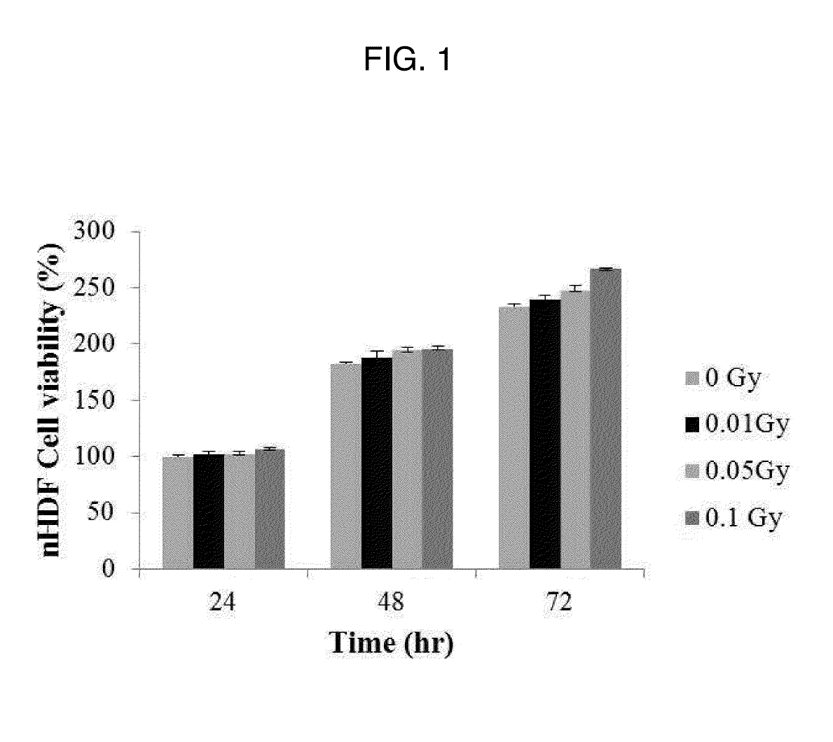

As a result of the analysis, as illustrated in FIG. 1, there was no significant change 24 hours after low-dose radiation, but the growth of cells were exhibited to be promoted in a low-dose radiation-dependent manner from 48 hours (an increase of 2.6%, 6.2%, and 7%, compared to that of control group), and there was large differences after 72 hours (an increase of 2.8%, 6.0%, and 14.2%, compared to that of control group). Accordingly, it was confirmed that the low-dose radiation promoted the growth of normal human dermal fibroblasts thus having excellent effect on skin improvement.

### Example 2

### Increase of Col1A1 Expression and Decrease of MMP1 Expression by Low-Dose Radiation

In order to confirm the effect of skin improvement by the irradiation of low-dose radiation, the present inventors measured the changes in expression of Col1A1, which is a representative collagen expressed by normal human dermal fibroblasts, and in the expression of MMP1, a collagen decomposing enzyme.

In order to confirm the level of collagen expression by the low-dose radiation, normal human dermal fibroblasts were irradiated with 0 Gy, 0.01 Gy, 0.05 Gy, and 0.1 Gy, and cultured for 24 hours. Then, the change in gene expression was measured via quantitative real time PCR (qRT-PCR), which continuously detects fluorescent materials, by labeling fluorescent materials to DNA products being amplified during the polymerase chain reaction (PCR) (Higuchi et al., 1992). In order to quantitatively confirm the change in expression of COL1A1 and MMP1 genes by the low-dose radiation, the resulting value was confirmed by the value of fluorescence emitted by the PCR products using SYBR green I (Invitrogen). The reaction solution was prepared by mixing 0.2 μM primers, 50 mM KCl, 20 mM Tris/HCl pH 8.4, 0.8 mM dNTP, 0.5 U Extaq DNA polymerase, 3 mM MgCl₂, 1×SYBR green in a PCR tube. Upon preparation of the reaction solution, it was subjected to primary denaturation at 94° C. for 30 seconds, and then denaturation, annealing, and polymerization were performed at 94° C. for 30 seconds, at 58° C. for 30 seconds, and at 72° C. for 30 seconds, for a total of 40 cycles, and the fluorescence intensity was measured upon termination of each cycle. The PCR results were verified via melting curves per each result. The threshold cycle (Ct) valued of each gene was standardized as Ct value of actin, and the amount of change in Ct value was compared and analyzed. Ct value refers to the number of cycles when the amount of fluorescence generated by PCR products (the number of amplified PCR products) reached a constant standard value, and the amount of gene expression can be confirmed via Ct values. The respective gene primers used in the experiments are listed in Table 1 below.

TABLE 1

| Gene | Forward primer | Reverse primer |
|---|---|---|
| β-actin | 5-GGATTCCTATGTGGGCGACGA-3 | 5-CGCTCGGTGAGGATCTTCATG-3 |
| | (SEQ ID NO: 1) | (SEQ ID NO: 2) |
| COL1A1 | 5-AGGGCCAAGACGAAGACATC-3 | 5-AGATCACGTCATCGCACAACA-3 |
| | (SEQ ID NO: 3) | (SEQ ID NO: 4) |
| MMP1 | 5-TCTGACGTTGATCCCAGAGAGCAG-3 | 5-CAGGGTGACACCAGTGACTGCAC-3 |
| | (SEQ ID NO: 5) | (SEQ ID NO: 6) |

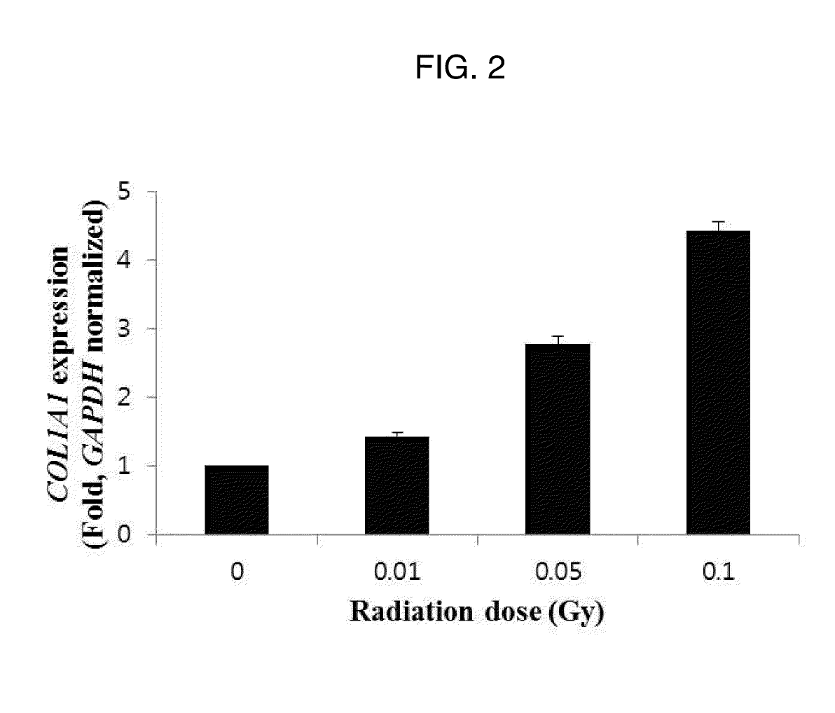

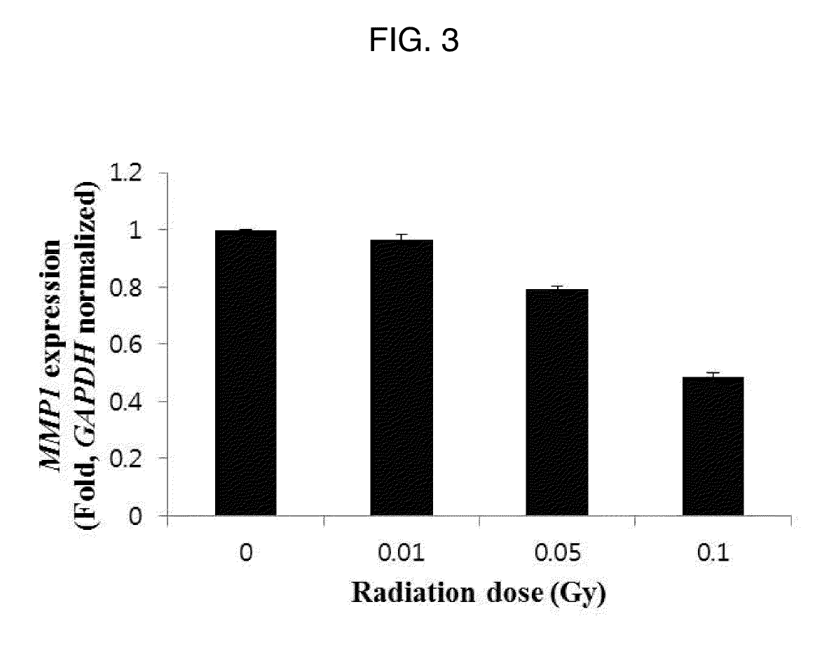

As a result of analysis, as illustrated in FIG. 2, the Col1A1 mRNA expression was increased by 1.4-, 2.8-, and 4.4-folds in a radiation dose-dependent manner. Accordingly, it was confirmed that the irradiation of the low-dose radiation promoted the expression of Col1A1 in normal human dermal fibroblasts. Additionally, as illustrated in FIG. 3, it was confirmed that the expression of MMP1 mRNA was reduced in a radiation dose-dependent manner by 3.4%, 20.7%, and 51.7%, compared to that of the control group.

### Example 3

### Effect of Improving Mouse Skin Wrinkles by Low-Dose Radiation

In order to examine the effect of skin wrinkle improvement by low-dose radiation, the present inventors analyzed the wrinkles of an aged mouse using a skin analyzer.

First, in order to confirm the effect of the low-dose radiation on skin wrinkles, aged SKH-1 mice purchased from Orientbio Inc. (Seongnam-si, Korea) and bred for more than 12 months were used. After irradiating 0 Gy, 0.01 Gy, 0.05 Gy, and 0.1 Gy of the low-dose radiation, the degree of wrinkles on the back of the mice two and four weeks after the irradiation was measured using PRIMOS Lite (field of view 4530-simple, flexible 3D measuring, GFMesstechnik GmbH, Germany) according to the device manual. The same test performer photographed the back areas of the mice three times consecutively after fixing the back to prevent the occurrence of contraction and relaxation.

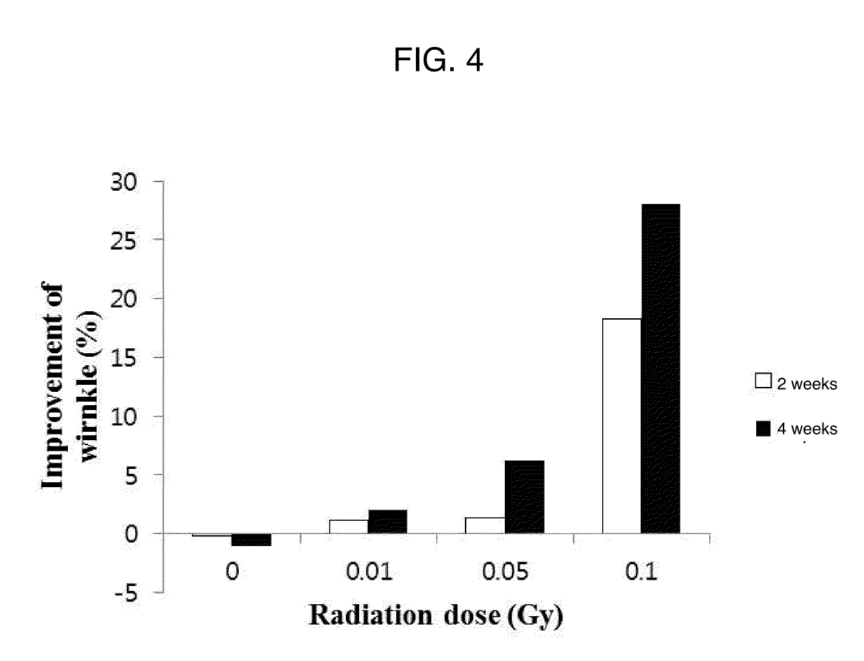

As a result of the analysis, as illustrated in FIG. 4, the control group exhibited a slight increase in wrinkles along with time, but there was an improvement in wrinkles in a radiation dose-dependent manner by irradiation of 0 Gy, 0.01 Gy, 0.05 Gy, and 0.1 Gy of the low-dose radiation, for example, by 1.15%, 1.35%, and 18.2% after two weeks, and 2.01%, 6.2%, and 28.1% after four weeks.

### Example 4

### Effect of Improving Mouse Skin Thickness by Low-Dose Radiation

It was confirmed in Examples 1 and 2 that the low-dose radiation increased the proliferation of dermal fibroblasts and the expression of collagen. Accordingly, the present inventors analyzed whether the thickness of dermal layer can be increased by the collagen produced thereof, and confirmed that the low-dose radiation has the effect of improving skin thickness.

First, aged SKH-1 mice purchased from Orientbio Inc. (Seongnam-si, Korea) were irradiated with 0 Gy, 0.01 Gy, 0.05 Gy, and 0.1 Gy of the low-dose radiation, respectively, and the thickness of dermal layer on the back of the mice were measured using DUB-Skin Scanner (tpm taberna promedicum, Germany), a skin ultrasound measuring device, two weeks and four weeks thereafter, according to the device manual. The DUB-Skin Scanner was applied with a gel for an ultrasound test, and the Probe was placed at a right angle to the skin, and the back of each mouse was pressed with the same pressure by the same test performer and measured.

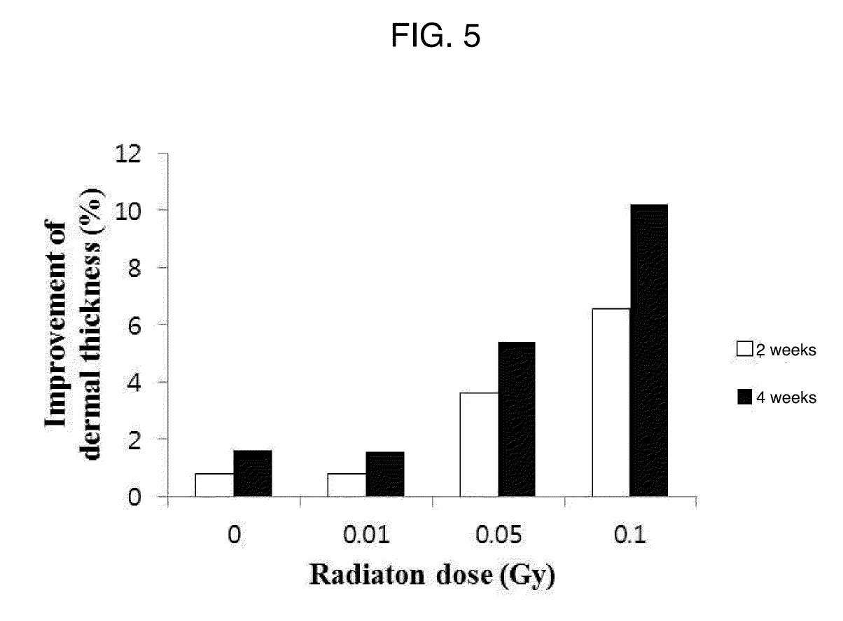

As a result of the analysis, as illustrated in FIG. 5, when irradiated with 0.01 Gy, there was no significant difference compared to that of the control group, but when irradiated with 0.05 Gy and 0.1 Gy, there was an improvement by 3.6% and 6.7% after two weeks, and by 5.4% and 10.2% after four weeks.

### Example 5

### Effect of Improving Mouse Skin Elasticity by Low-Dose Radiation

In order to confirm the effect of the low-dose radiation on improving skin elasticity, the present inventors performed experiments using aged mice.

First, aged SKH-1 mice purchased from Orientbio Inc. (Seongnam-si, Korea) were irradiated with 0 Gy, 0.01 Gy, 0.05 Gy, and 0.1 Gy of low-dose radiation, respectively, and the changes in skin elasticity on the back of the mice were measured using DermaLab USB elasticity probe (Cortex Technology, Inc., Denmark). The DermaLab USB elasticity probe indicates changes in skin and restoring force according to the inhalation on the measurement area and the duration of inhalation in terms of values, and the same test performer fixed the probe on the back of the mice with tape and measured three times.

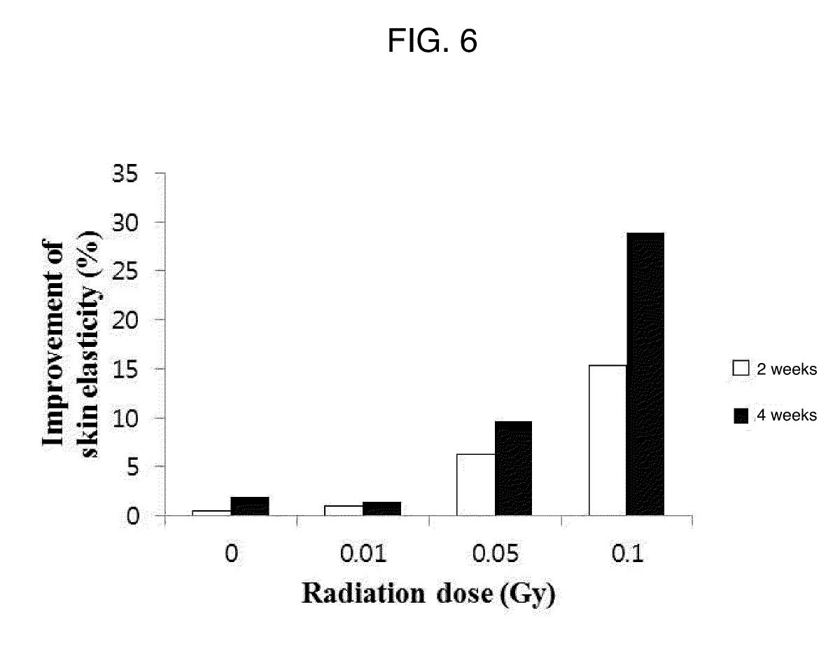

As a result, as illustrated in FIG. 6, there was no significant change in skin elasticity when irradiated with 0.01 Gy compared to the control group two weeks and four weeks after the irradiation, but when irradiated with 0.05 Gy and 0.1 Gy the skin elasticity was improved by 6.3% and 15.3% after two weeks, and 9.6% and 28.8% after four weeks.

### Example 6

### Effect of Promoting Keratinocytes by Low-Dose Radiation

The present inventors confirmed the effect of promoting keratinocytes by irradiation of low-dose radiation on HaCaT cells.

HaCaT cells were purchased from Cell Line Service (Germany) for use. For a cell culture solution, KGM gold keratinocyte growth medium (Lonza, Basel, Switzerland) was used. Culture conditions were 37° C., 95% humidity, and 5% CO₂ incubator.

First, in order to examine the level of cytotoxicity generated, HaCaT cells were irradiated with 0 Gy, 0.01 Gy, 0.05 Gy, and 0.1 Gy, cultured for 24, 48, and 72 hours, and the viability of the cells were measured.

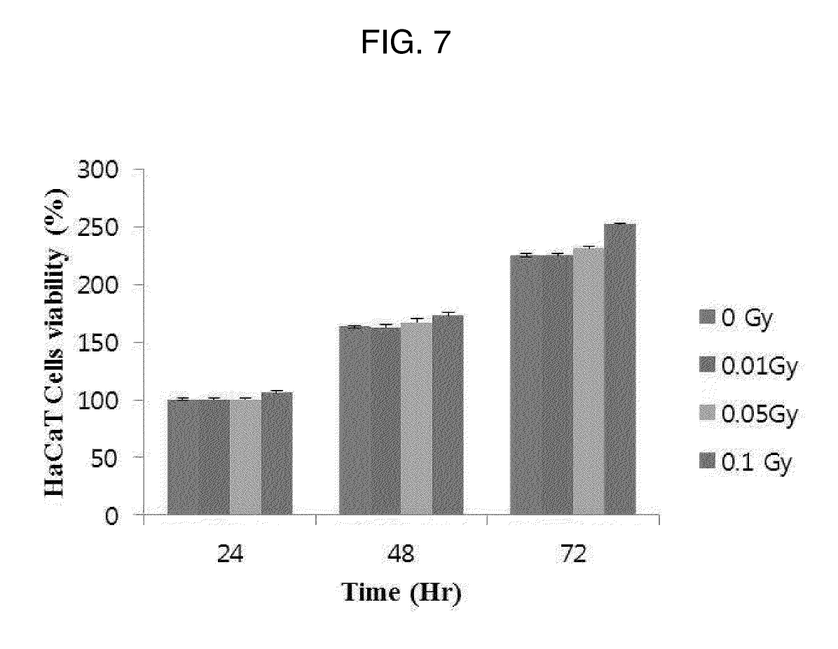

The result revealed that, 24 hours after the irradiation of 0.1 Gy, there was about 6% increase of cell growth but there was no significant change when irradiated at a lower amount of radiation. Meanwhile, 48 hours after the irradiation of 0.05 Gy, and 0.1 Gy, there were 2.3% and 6.0% of cell growth, and 72 hours after the irradiation of 0.05 Gy, and 0.1 Gy, there were 2.6% and 11.9% of cell growth (FIG. 7). Accordingly, it was confirmed that low-dose radiation can promote the growth of keratinocytes.

### Example 7

### Effect of Promoting Differentiation of Keratinocytes by Low-Dose Radiation

In order to confirm the effect of low-dose radiation on the differentiation of keratinocytes, the HaCaT cells irradiated with low-dose radiation were treated with CaCl₂ to induce differentiation, and the degree of differentiation according to time zone was examined using differentiation markers.

Involucrin begins to be synthesized in a spinous layer, forms keratinized epithelial cells in a horny layer to protect keratinocytes, and keratin 1 and keratin 10 are specific keratins expressed both in the spinous layer and the horny layer, and also representative biomarkers.

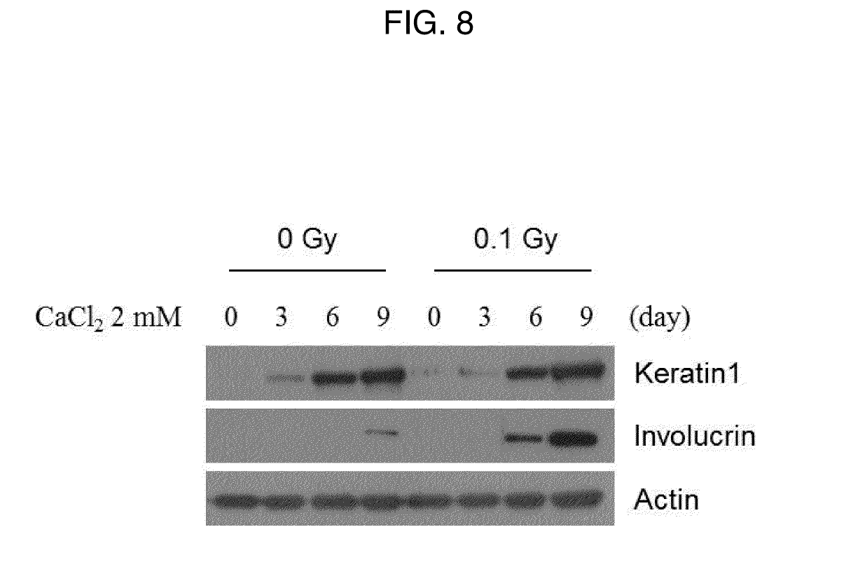

As a result, as illustrated in FIG. 8, there was no significant change in keratin 1 when irradiated with low-dose radiation, whereas involucrin began to exhibit a significant change from the sixth day after inducing differentiation. This indicates that low-dose radiation does not influence much on the initial stage of differentiation of keratinocytes but promote differentiation from the intermediate stage thereon.

### Example 8

### Effect of Recovering Mouse Skin Barriers by Low-Dose Radiation

Because the growth and differentiation of keratinocytes were exhibited to increase by low-dose radiation in Examples 6 and 7, the present inventors performed the following experiments in order to evaluate the effect of low-dose radiation on the recovery of skin barriers.

First, SKH-1 mice purchased from Orientbio Inc. (Seongnam-si, Korea) were periodically applied with acetone to artificially damage skin barriers, and irradiated with low-dose radiation of 0.1 Gy. Then, the Trans Epidermal Water Loss (TEWL) of the area, where the acetone was applied, was measured using DermaLab USB TEWL probe (Cortex Technology, Inc., Denmark) according to the device manual.

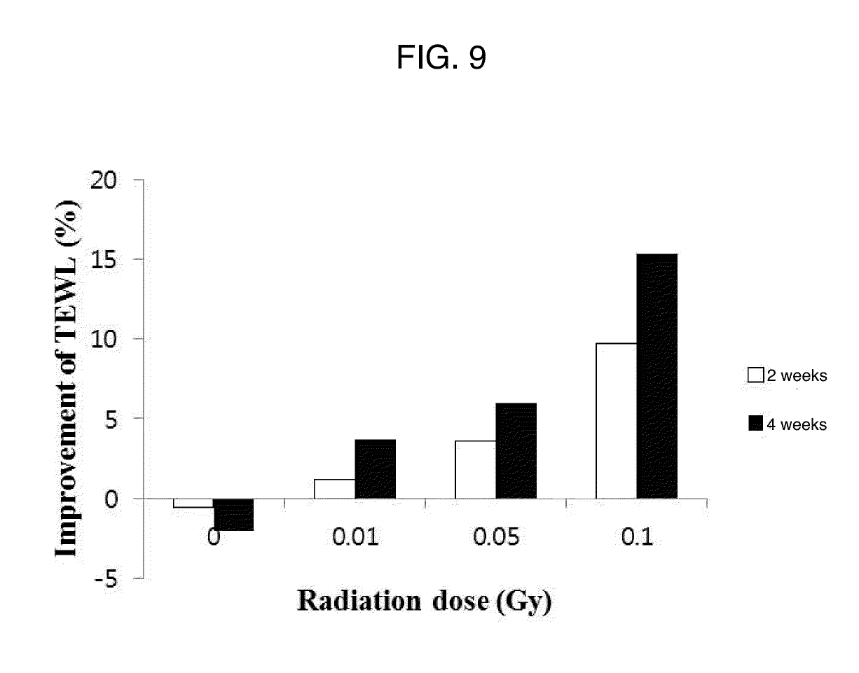

As a result, as illustrated in FIG. 9, it was confirmed that the Trans Epidermal Water Loss was gradually decreased in the control group two weeks and four weeks after the irradiation, whereas, when irradiated with low-dose radiation of 0 Gy, 0.01 Gy, 0.05 Gy, and 0.1 Gy, there was an improvement of 1.17%, 3.6%, and 9.7%, respectively, two weeks after the irradiation, and an improvement of 3.7%, 6.0%, and 15.3%, respectively. Accordingly, it is suggested that the irradiation of low-dose radiation on skin promoted the differentiation of the horny layer thereby more rapidly accomplishing recovery of skin barriers.

### Example 9

### Effect of Improving Mouse Skin Texture by Low-Dose Radiation

Based on the effect of improving the recovery of skin barriers confirmed in Example 8, the present inventors examined whether the irradiation of low-dose radiation also has the effect of improving the skin texture.

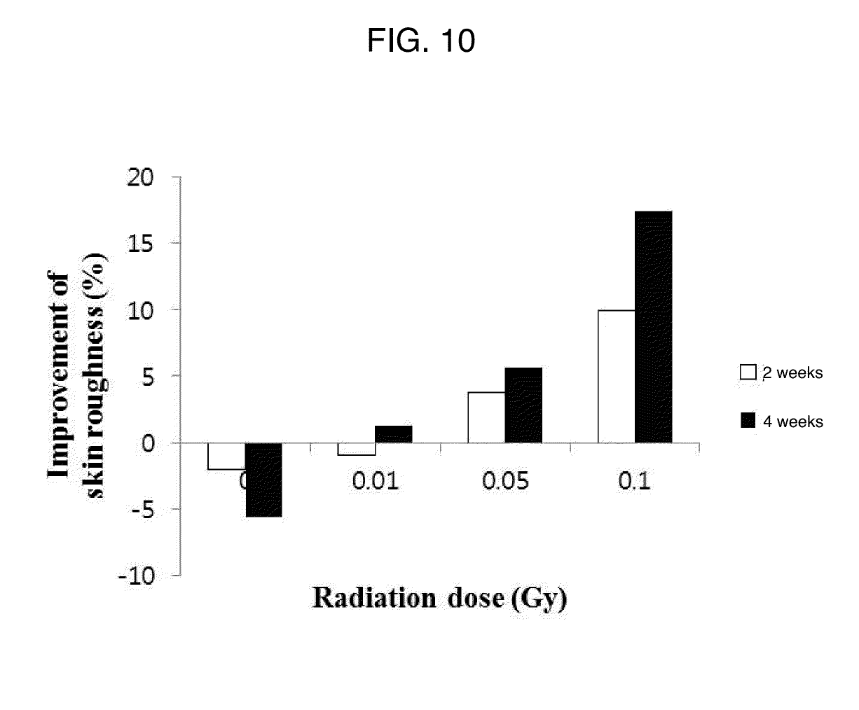

First, SKH-1 mice purchased from Orientbio Inc. (Seongnam-si, Korea) were irradiated with low-dose radiation of 0 Gy, 0.01 Gy, 0.05 Gy, and 0.1 Gy, and the change in their skin roughness was analyzed using PRIMOS Lite (field of view 18×13-simple, flexible 3D measuring, GFMesstechnik GmbH, Germany) (FIG. 10). The same test performer photographed the back areas of the mice three times consecutively after fixing the back to prevent the occurrence of contraction and relaxation.

As a result, the control group exhibited an increase in skin roughness two weeks and four weeks after the irradiation, whereas the experimental groups irradiated with low-dose radiation exhibited an improvement in the skin roughness in a radiation dose-dependent manner. From these results, it was confirmed that low-dose radiation promoted the growth and differentiation of keratinocytes, and as a result, rapidly promoting the recovery of skin barriers and also having the effect of improving skin texture.

### Example 10

### Effect of Promoting Growth of Human Dermal Papilla Cells by Low-Dose Radiation

In order to confirm the effect of low-dose radiation on promoting hair growth by low-dose radiation, the present inventors irradiated low-dose radiation on human dermal papilla cells.

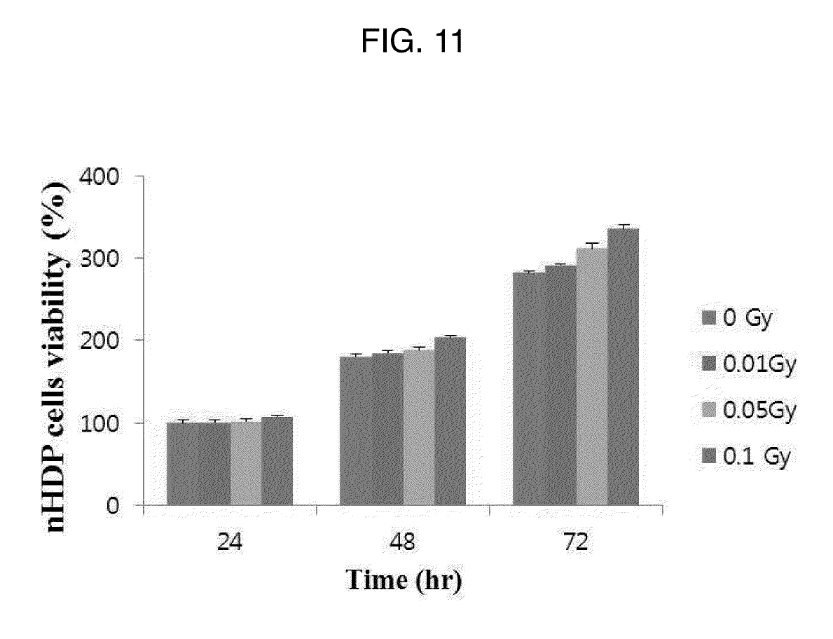

First, in order to examine the level of cytotoxicity generated, the cells were irradiated with 0 Gy, 0.01 Gy, 0.05 Gy, and 0.1 Gy, cultured for 24, 48, and 72 hours, and the viability of the cells were measured. There was an increase of cell growth of about 2.0% with 0.05 Gy radiation, about 7.9% with 0.1 Gy radiation, 24 hours after the irradiation, compared to that of the control group, whereas there was no significant change with 0.01 Gy radiation. When irradiated with 0.01 Gy, 0.05 Gy, and 0.1 Gy, 48 hours after the irradiation, there was an increase in cell growth of 2.3%, 4.2%, and 12.7%, compared to that of the control group, respectively, and 72 hours after the irradiation, there was an increase in cell growth of 3.3%, 10.3%, and 18.9%, respectively (FIG. 11).

Accordingly, it was confirmed that low-dose radiation has the effect of promoting the growth of human dermal papilla cells thus having the effect of promoting hair growth.

### Example 11

### Measurement of the Effect of Mouse Hair Growth Capability by Low-Dose Radiation

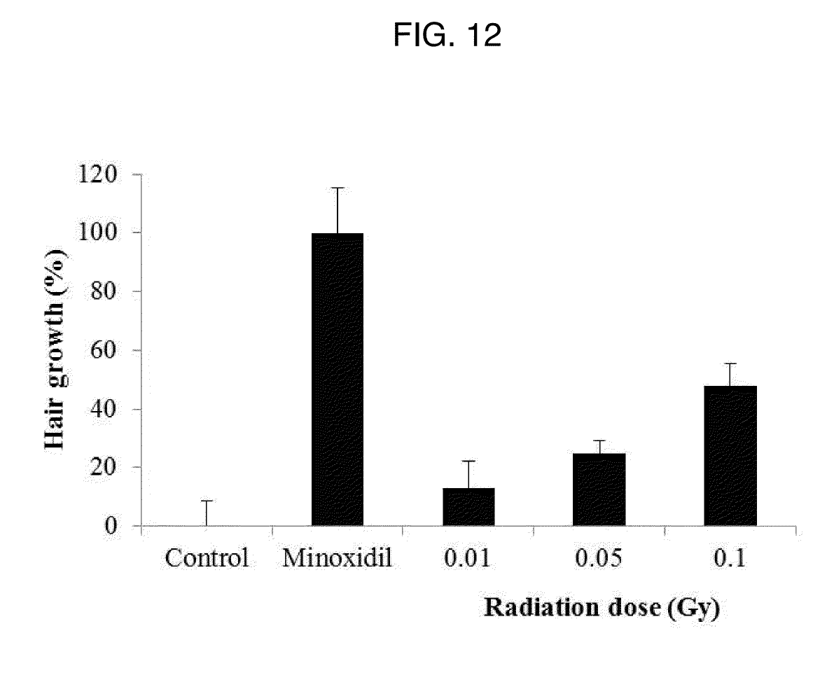

In order to confirm the effect of low-dose radiation on hair growth capability, the present inventors irradiated low-dose radiation on C57BL/6 mice and examined the change in their hair growth capability (FIG. 12).

C57BL/6 mice were purchased from Orientbio Inc. (Seongnam-si, Korea). The mice have a characteristic hair cycle, where the hairs enter the regressing phase from the 6th week after birth, become resting phase from the 7th week, and return to growth phase from the 12^{th} week, and are most commonly used as in vivo animal models for hair growth test. Six-week old C57BL/6 female mice were purchased, allowed to adapt for a week, and subjected to experiments when they became 7-week old. The mice were anesthetized and the hairs on their back were removed using a hair remover. One day thereafter, those mice which had no injury on the back were subjected to low-dose radiation. Four weeks after the irradiation, the mice were photographed, and the color change in the irradiated area were digitized using an image analyzer, and the improvement rate was calculated based on Minoxidil® as 100%.

As a result, as illustrated in FIG. 13, it was confirmed that the hair growth capability was increased 13%, 25%, and 48%, with the irradiation of increasing amount of radiation of 0 Gy, 0.01 Gy, 0.05 Gy, and 0.1 Gy, compared to that of the control group, thus confirming that low-dose radiation has the effect of promoting hair growth.

The present disclosure has been described with reference to preferred embodiments. From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

What is claimed is:

1. A method for improving condition of skin comprising irradiating low-dose radiation to the skin of a subject.

2. The method of claim 1, wherein the low-dose radiation is at least one selected from the group consisting of α-ray, β-ray, γ-ray, electron-ray, UV-ray, and X-ray.

3. The method of claim 1, wherein the low-dose radiation is irradiated ranging from 0.01 Gy to 0.2 Gy.

4. The method of claim 1, wherein the low-dose radiation is irradiated ranging from 0.01 Gy to 0.1 Gy.

5. The method of claim 1, wherein the low-dose radiation is irradiated ranging from 0.05 Gy to 0.1 Gy.

6. The method of claim 1, wherein, in irradiating the low-dose radiation, the low-dose radiation is exposed once or multiple times at predetermined time intervals.

7. The method of claim 1, wherein, in irradiating the low-dose radiation, the method is characterized in that the growth of normal human dermal fibroblasts is promoted, the collagen synthesis within the normal human dermal fibroblasts is increased, or the metalloproteinase synthesis within the normal human dermal fibroblasts is decreased.

8. The method of claim 1, wherein, in irradiating the low-dose radiation, the method is characterized in that the growth of keratinocytes is promoted or the differentiation of keratinocytes is promoted.

9. The method of claim 1, wherein the skin improvement is selected from the group consisting of decrease of skin wrinkles, increasing recovery of skin barriers, increasing skin thickness, increasing skin elasticity, and increasing skin moisture.

10. A method of promoting hair growth comprising irradiating skin of as subject with low-dose radiation.

11. The method of claim 10, wherein the low-dose radiation is at least one or more selected from the group consisting of α-ray, β-ray, γ-ray, electron-ray, UV-ray, and X-ray.

12. The method of claim 10, wherein the low-dose radiation is irradiated ranging from 0.01 Gy to 0.2 Gy.

13. The method of claim 10, wherein the low-dose radiation is irradiated ranging from 0.01 Gy to 0.1 Gy.

14. The method of claim 10, wherein the low-dose radiation is irradiated ranging from 0.05 Gy to 0.1 Gy.

15. The method of claim 10, wherein, in irradiating the low-dose radiation, the growth of human dermal papilla cells is promoted or hair follicle cells are activated.
